(19) United States
(12) Patent Application Publication
(10) Pub. No.: US 2023/0338246 A1
(43) Pub. Date: Oct. 26, 2023

(54) ANTIPERSPIRANT STICK COMPOSITIONS

(71) Applicant: Conopco, Inc., d/b/a UNILEVER, Englewood Cliffs, NJ (US)

(72) Inventors: Nigel John Cross, Pontefract (GB); Bruce Steven Emslie, Leeds (GB); Craig James Luckwell, Leeds (GB)

(21) Appl. No.: 18/004,997

(22) PCT Filed: Jul. 9, 2021
(86) PCT No.: PCT/EP2021/069172

§ 371 Date: Jan. 10, 2023

(30) Foreign Application Priority Data
Jul. 13, 2020 (EP) 20185487.4

(51) Int. Cl. A61K 8/26; A61K 8/02; A61K 8/06; A61K 8/34; A61Q 15/00

(52) U.S. Cl. CPC A61K 8/26; A61K 8/0229; A61K 8/064; A61K 8/345; A61Q 15/00

(57) ABSTRACT

A solid antiperspirant stick composition in the form of a water-in-oil emulsion comprising (i) an aqueous phase comprising dissolved aluminium sesquichlorohydrate antiperspirant active; (ii) an oil phase comprising a solidifying agent; (iii) an emulsifier and (iv) at least 5% by weight of a polyol selected from glycerol, propylene glycol and/or PEG oligomers having a molecular weight of from 180 to 420.

### FIELD OF INVENTION

The present invention relates to solidified, water-in-oil emulsion antiperspirant stick compositions and their methods of manufacture, especially to such compositions containing a highly effective astringent antiperspirant salt and a polyol.

### BACKGROUND

Antiperspirant stick compositions are typically characterised by being integral and self-supporting, commonly in the form of a rod or bar, usually dispensed from a tubular container having an open end and provided with a platform that is moveable towards the open end. Some antiperspirant stick compositions are anhydrous, whilst others are aqueous compositions in the form of an emulsions. In such emulsion compositions, the antiperspirant active is commonly dissolved in the aqueous phase which is typically dispersed in an oil continuous phase to form a water-in-oil emulsion.

Emulsion stick formations are disclosed in a series of patents to Unilever, including US 6287544, US 6455056, US 6248312 and WO 2003/059307. These specifications disclose that emulsion sticks can be made using structurants, many of which are fibre-forming small molecule gelling agents. A combination of structurants has been disclosed in WO 2004/098551, also to Unilever.

Antiperspirant water-in-oil emulsions are also disclosed in EP 1280502 in which the oil phase is structured with an ester wax.

A water-in-oil emulsion stick comprising potassium alum in the aqueous phase and in which the oil phase is gelled with polyethylene is disclosed in USP 6139824.

### SUMMARY OF THE INVENTION

The present invention relates to antiperspirant stick formulations which comprise droplets of a solution of a highly effective antiperspirant active dispersed in a continuous oil phase, such formulations being made solid by the presence of a solidifying agent.

The present invention addresses a problem found with the antiperspirancy performance of antiperspirant stick compositions that are in the form of water-in-oil emulsions. The antiperspirant active is present in the dispersed aqueous phase in such sticks, locked within the oily continuous phase. It is hypothesized that this is can lead to a reduced speed and efficiency of delivery of the antiperspirant active to the skin surface following topical application, the oily continuous phase, thickened by the solidifying agent, forming a barrier layer between the skin and the antiperspirant active. Such difficulties are accentuated in water-in-oil emulsion sticks also comprising a polyol as required for sensory benefits in the stick compositions of the present invention. Such polyols are present in the dispersed aqueous phase of the emulsion and, without wishing to be bound by theory, they can further reduce the speed and efficiency of release of the antiperspirancy active to the skin surface, whether by increasing the viscosity of the aqueous phase and/or by direct interaction with antiperspirant active. This problem is further accentuated when the polyol is present at a high level within the aqueous phase and/or within the composition as a whole.

A further benefit of the present invention is that relatively high levels of polyol, in particular glycerol, can be incorporated without making the stick too sticky on and after application. As the level of AP active can also contribute to the feeling of stickiness, the present invention benefits from only needing a relatively low level of this component to achieve good antiperspirancy, such as from 10 to 20% by weight, for example.

It is an object of the present invention to provide a water-in-oil antiperspirant stick formulation that exhibits high antiperspirancy efficacy, superior to water-in-oil antiperspirant stick formulation comprising conventional aluminium zirconium chlorohydrex salts, for example.

It is a further object of the present invention to provide a water-in-oil antiperspirant stick formulation that exhibits desirable sensory properties either on application or thereafter.

It is a further object of the present invention to provide a water-in-oil antiperspirant stick formulation that improves the quality of the skin, whether assessed visually and/or by touch, following its topical application.

It is a yet another object of certain of the embodiments of the invention to provide a water-in-oil antiperspirant stick formulation which results in reduced visible marks on clothing following application. Such visible marks are typically seen as white staining, on fabrics worn in proximity to the site of application of the composition. The benefit is particularly significant at low AP active levels, such as 10 to 20% by weight of the total composition, such levels giving low white marks and yet good antiperspirancy performance.

According to a first aspect of the invention, there is provided a solid antiperspirant stick composition in the form of a water-in-oil emulsion comprising: an aqueous phase comprising dissolved aluminium sesquichlorohydrate (ASCH) antiperspirant active; an oil phase; an emulsifier; a polyol and a solidifying agent; wherein the polyol is glycerol, propylene glycol and/or PEG oligomers having a molecular weight (weight average) of from 180 to 420 and is present at a total level of at least 5% by weight of the total composition.

According to a second aspect of the invention, there is provided a process for the preparation of an antiperspirant stick composition in accordance with the first aspect of the invention.

According to a third aspect of the invention there is provided a non-therapeutic method of reducing perspiration comprising the topical application of a stick composition according to the first aspect of the invention, particularly in the underarm. This method may also deliver skin care benefits, particularly for those with sensitive skin.

According to a fourth aspect of the invention there is provided a non-therapeutic method of reducing body malodour comprising the topical application of a stick composition according to the first aspect of the invention, particularly in the underarm. This method may also deliver skin care benefits, particularly for those with sensitive skin.

According to a fifth aspect of the invention there is provided a non-therapeutic method of providing skin care benefits comprising the topical application of a stick composition according to the first aspect of the invention, particularly in the underarm.

### DETAILED DESCRIPTION

Herein, features expressed as “preferred” with regard to a particular aspect of the invention should be understood to be preferred with regard to each aspect of the invention (likewise, features expressed as “more preferred” or “most preferred”).

Herein, preferred features of the invention are particularly preferred when used in combination with other preferred features.

Herein, “ambient conditions” refer to 20° C. and 1 atmosphere pressure, unless otherwise indicated.

Herein, all percentages, ratios, parts and amounts are by weight, unless otherwise indicated.

Herein, amounts and concentrations of ingredients are percentages by weight of the total composition, unless otherwise indicated.

Herein, references to amounts of components such as “liquid oil” or “solidifying agent” relate to the total amount of such components present in the composition.

Herein, the word “comprising” is intended to mean “including” but not necessarily “consisting of”, i.e., it is non-exhaustive.

Herein, “cosmetic” methods and compositions should be understood to mean non-therapeutic methods and compositions, respectively.

Herein, an “aluminium-based” antiperspirant (AP) salt is one comprising aluminium.

Herein, a “calcium-activated” AP salt is one that has been heated in water with a water-soluble calcium salt during its manufacture.

Herein, “solidified” refers to a state of matter incapable of flow at a temperature of 25° C. and a pressure of 1 atmosphere.

Herein, the “hardness” of stick and soft solid compositions refers to the depth, in millimetres, that a cone penetrates into a test specimen under fixed conditions, as determined in accordance with the procedures of ASTM Method D217-48, incorporated herein by reference, using a Petrotest PNR10 Penetrometer (or equivalent), equipped with an ASTMD2884 plunger (Petrotest Cat. #18-0081 or equivalent, weight =47.5 g) and a 2.5 g aluminium cone, 20° angle with a base diameter of 10 mm, wherein hardness values are reported as an average of 6 replicate measurements.

Compositions according to the present invention typically have a hardness of less than 20 mm, preferably less than 15 mm and more preferably less than 10 mm. Such sticks can demonstrate a combination of aesthetically desirable properties, including sensory benefits such as the avoidance of excessive drag on application and sometimes lower visible marks on clothing worn in close proximity to the area of application on the skin.

The aqueous phase of compositions of the invention comprises an aqueous solution of an ASCH AP active and a polyol, as described further hereinbelow. It may also comprise other water-soluble materials, as described further hereinbelow.

Herein, water-soluble means having a solubility in water of at least 10 g/l at 25° C.

The aqueous phase typically comprises from 30 to 68% of the total composition. Preferably, it comprises from 40 to 65% and more preferably from 45 to 60% of the total composition.

The aqueous phase comprises an ASCH AP active as an essential component. The ASCH AP active has the chemical formula of Al₂OH_{4.4}Cl_{1.6} to Al₂OH_{4.9}CI_{1.1}. Most commercial ASCH samples are of chemical formula Al₂OH_{4.7}Cl_{1.3} to Al₂OH_{4.9}Cl_{1.1} and it is preferred to use actives of this formula.

Particularly preferred AP actives comprise calcium and particularly preferred actives also comprise an amino acid, in particular glycine. Such actives are described in WO 2014/187685 (Unilever, 2014). The calcium is typically added in the form of calcium chloride. It is especially preferred that such actives are calcium-activated ASCH salts.

In the preferred ASCH AP salts comprising calcium, the molar ratio of calcium to aluminium is typically at least 1:40, preferably at least 1:30 and more preferably at least 1:20. It is not advantageous to have the calcium concentration in excess of the aluminium concentration, indeed it is preferred that the calcium concentration is no more than half that of the aluminium concentration and more preferred that it is no more than a fifth of said concentration. For the preferred molar ratios of calcium to aluminium of at least 1:40 and at least 1:20, it is independently preferred that this ratio is no greater than 1:2 and more preferred that it is no greater than 1:5. In particularly preferred embodiments, the molar ratio of calcium to aluminium is at least 1:15 and preferably no greater than 1:5 and in especially preferred embodiments it is at least 1:12 and preferably no greater than 1:5.

Herein, references to molar amounts and ratios of “aluminium” are calculated on the basis of mono-nuclear aluminium, but include aluminium present in poly-nuclear species; indeed, most of the aluminium in the salts of relevance is present in poly-nuclear species.

AP actives that are complexes with glycine are preferred, particularly when they also comprise calcium and/or are activated by calcium. In such AP actives, the molar ratio of amino acid to aluminium is preferably at least 1:20, more preferably at least 1:15 and most preferably at least 1:10. It is not advantageous to have the amino acid concentration in excess of the aluminium concentration; hence, the molar amino acid to aluminium is preferably from 1:20 to 1:1, more preferably from 1:15 to 1:2 and most preferably from 1:10 to 1:5.

In certain especially preferred embodiments, a calcium-activated ASCH glycine complex is used in which the molar ratio of calcium to aluminium is from 1:15 to 1:5 and the molar ratio of amino acid to aluminium is also from 1:15 to 1:5.

The proportion of antiperspirant salt is calculated excluding the weight of any water of hydration but including any complexing agent that may be present, such as glycine and any activating agent, such as calcium chloride.

The weight concentration of the antiperspirant salt in the aqueous phase is commonly not higher than 50% and is advantageously at least 10%. In preferred embodiments its weight concentration is from 15 to 40% and in more preferred embodiments it is from 20 to 30%.

The weight of antiperspirant salt in the entire composition is usually at least 6% and is commonly up to 30%. The range of antiperspirant salt concentration for the composition is preferably from 8% to 25% and more preferably from 10 to 20%.

In addition to water and AP active, the aqueous phase comprises one or more polyols selected from glycerol, propylene glycol and/or PEG oligomers having a molecular weight (weight average) of from 180 to 420. Of these polyols, glycerol is the most preferred. The polyol is present at a total level of at least 5% by weight of the total composition and helps deliver the desirable sensory properties of the stick compositions of the present invention. The polyhydric alcohol is understood to counteract the astringency of the AP active, by acting as a humectant. Such materials may be advantageously employed at from 5% to 20%, preferably at from 10 to 20% and more preferably at from 10 to 15% of the total composition. Other desirable water-soluble components that may be incorporated in compositions of the invention include licorice extract and/or vitamins, such as vitamin B3 (niacinamide) and vitamin C, that may enhance skin tone following topical application of the composition of the invention. Vitamins tend to be particularly sensitive to elevated temperature and the compositions of the invention are particularly well suited to their incorporation due to the relatively low melting point of the solidifying agent employed.

The oil phase of the compositions of the invention comprises a liquid oil and a solidifying agent.

The oil phase typically comprises from 30 to 70% of the total composition. Preferably, it comprises from 35 to 60% and more preferably from 40 to 55% of the total composition. For the purpose of calculating the amount of the oil phase, the emulsifier is considered to be a component of the oil phase.

The liquid oil may be chosen from any of wide range of such oils. In some embodiments the oil phase may comprise one or more oils selected from volatile silicone oils, ester oils, ether oils or mineral oils.

Herein, the liquid oil is a water-immiscible oil having a melting point of 25° C. or less. Preferably, it has a melting point of 20° C. or less. In some embodiments, the liquid oil has a melting point of from -50° C. to 25° C. and preferably from -30° C. to 20° C.

The oil phase may comprise more than one liquid oil as defined hereinabove. In such circumstance, amounts and ratios referring to “liquid oil” relate to all such oils present.

In some embodiments of the invention, the oil phase comprises one or more volatile silicone oils. By volatile herein is meant having a measurable vapour pressure at 20 or 25° C. Typically, the vapour pressure of a volatile silicone oil lies in a range from 1 or 10 Pa to 2 kPa at 25° C. Volatile silicone oils can be linear or cyclic siloxanes, usually containing from 3 to 9 silicon atoms, and commonly from 4 to 6 silicon atoms, the silicon atoms being substituted by methyl groups. It is especially desirable to employ volatile silicone oils in which at least 80% and particularly at least 90% contain at least 5 silicon atoms, such as cyclopentadimethylsiloxane (D5), cyclohexadimethylsiloxane (D6), dodecamethylpentasiloxane and tetradecamethylhexasiloxane. Such oils are highly desirable for many consumers because they can evaporate without causing undue skin cooling. In some embodiments, the volatile silicone oils may comprise at least 30% of the oil phase and normally not higher than 95% thereof. In a number of desirable compositions their weight proportion in the oil phase is up to 60%, and a preferred range in the oil phase is from 30 to 55%.

The liquid oil can alternatively or additionally comprise one or more non-volatile oils, which can be silicone oils and/or non-silicone oils. Non-volatile silicone oils employed herein preferably contain one or more unsaturated substituents such as phenyl or diphenylethyl in replacement of the corresponding number of methyl substituents in polycyclosiloxanes or more preferably in linear siloxanes, often having 2 or 3 silicon atoms. Such non-volatile oils have a higher refractive index than that of the volatile silicone oils and tend to mask the antiperspirant active when it is deposited on skin. The non-volatile oils can also comprise dimethiconols, which are hydroxyl-terminated. The proportion of non-volatile silicone oils in the oil phase is commonly up to 25% such as from 0.25 to 20% of the phase. In some embodiments, the oil phase comprises from 1 to 10% of the non-volatile silicone oil, and in other chosen embodiments, the non-volatile silicone oil provides from 10 to 20% of the oil phase.

The weight ratio of volatile to non-volatile oils is desirably from 2:1 to 1:2, and particularly from 3:2 to 2:3.

The oil phase can alternatively or additionally comprise one or more hydrocarbon fluids, which can be either volatile or non-volatile. Suitable hydrocarbon fluids include liquid aliphatic hydrocarbons such as mineral oils or hydrogenated polyisobutene, desirably selected to exhibit a low viscosity. Further examples of liquid hydrocarbons are polydecene and paraffins and isoparaffins of at least 10 carbon atoms. Hydrocarbon fluids conveniently comprise from 0 to 25% of the oil phase.

In at least some advantageous embodiments, the oil phase comprises liquid aliphatic or aromatic ester oils. Such oils can act as emollients and in addition can affect the sensory attributes of the resultant emulsion. Suitable aliphatic esters contain at least one long chain alkyl group, such as esters derived from C₁ to C₂₀ alkanols esterified with a C₈ to C₂₂ alkanoic acid or C₆ to C₁₀ alkanedioic acid. The alkanol and acid moieties or mixtures thereof are preferably selected such that they each have a melting point of below 20° C. Aliphatic esters include isopropyl myristate, lauryl myristate, isopropyl palmitate, diisopropyl sebacate and diisopropyl adipate. Further and very suitable ester oils include glyceride oils and in particular triglyceride oils derived from glycerol and fatty acids containing at least 6 carbons and especially natural oils.

It is especially desirable to employ aromatic ester oils compared, for example with aliphatic ester oils, in view of their physical properties, such as refractive index. Suitable liquid aromatic esters include fatty alkyl benzoates. Examples of such esters include suitable C₈ to C₁₈ alkyl benzoates or mixtures thereof, including in particular C₁₂ to C₁₅ alkyl benzoates e.g. those available under the trademark Finsolv TN. Such benzoate esters are especially desirable for employment in an aqueous antiperspirant emulsion, and one employing a wax to gel the oil phase, relative to alkyl mono or diesters mentioned above, by virtue of their properties that promote superior aesthetic attributes. An aryl benzoate, such as benzyl benzoate can also be used. Yet other suitable ester oils include oils in which a short alkylene group of 1 to 3 carbons, optionally substituted by a methyl group, is interposed between benzene and benzoate residues. It is advantageous to select an ester oil having a refractive index of at least 1.47.

The total proportion of ester oils, including both aliphatic and aromatic ester oils (but excluding any fragrance oil) is often from 0 to 50% of the oil phase, is desirably at least 5% and with benefit comprise at least 20%. Their total proportion is in many embodiments desirably from 25% up to 40% of the oil phase. The weight ratio of aromatic ester oil to aliphatic ester oil is often selected in the range of from 1:2 to 20:1, such from 1:1·to 8:1.

Natural ester oils which desirably can be employed herein comprise one or more unsaturated C18 fatty acid glycerides. In many instances, such ester oils comprise one or more triglycerides. The fatty acid residues in the oils can comprise, commonly, from one to three olefinic unsaturated bonds and often one or two. Whilst in many instances the olefinic bonds adopt the trans configuration, in a number of desirable products the bond or bonds adopt the cis configuration. If two or three olefinic unsaturated bonds are present, they can be conjugated. The fatty acid can also be substituted by an hydroxyl group. The natural oils employable herein desirably comprise one or more triglycerides of oleic acid, linoleic acid, linolenic acid or ricinoleic acid. Various isomers of such acids often have common names, including linolenelaidic acid, trans 7-octadecenoic acid, parinaric acid, pinolenic acid punicic acid, petroselenic acid and stearidonic acid. It is especially desirable to employ glycerides derived from oleic acid, linoleic acid or petroselenic acid, or a mixture containing one or more of them.

Natural oils containing one or more of such triglycerides include coriander seed oil for derivatives of petroselinic acid, impatiens balsimina seed oil, parinarium laurinarium kernel fat or sabastiana brasilinensis seed oil for derivatives of cis-parinaric acid, dehydrated castor seed oil, for derivatives of conjugated linoleic acids, borage seed oil and evening primrose oil for derivatives of linoleic and linolenic acids, aquilegia vulgaris oil for columbinic acid and sunflower oil, olive oil or safflower oil for derivatives of oleic acid, often together with linoleic acids. Other suitable oils are obtainable from hemp, such as stearadonic acid derivatives and maize corn oil. An especially preferred oil, which is a natural oil, is sunflower seed oil.

The proportion of the natural oil, viz particularly the triglyceride oils of unsaturated fatty acids, if present in the composition, is often selected in the range of from 0.1 to 10% of the oil phase, desirably at least 0.5% and in some embodiments particularly in the range of up to 8% of the oil phase.

Some preferred embodiments employ a natural ester oil in the oil phase together with a water-soluble humectant, such as those mentioned hereinbefore, in the aqueous phase, and particularly in a weight ratio to each other of from 3:1 to 1:3.

A further class of particularly suitable oils comprises a) non-volatile liquid aliphatic ethers derived from at least one fatty alcohol that desirably contains at least 10 carbon atoms, such as myristyl ether derivatives e.g. PPG-3 myristyl ether or lower alkyl (≤ C₆) ethers of polyglycols (preferably polypropylene glycol and especially 10 to 20 units, such as an ether named as PPG-14 butyl ether in the CTFA. The class of non-volatile ethers additionally comprises b) dialkyl ethers derived from C8 or C10 linear aliphatic alcohols, and particularly dioctyl ether. The invention emulsions herein can alternatively or additionally comprise a dialkyl carbonate derived from C8 or C10 linear aliphatic alcohols, and particularly dioctyl carbonate. It is often convenient for the aliphatic ether or dialkyl carbonate to constitute at least 0%, and especially at least 10%, particularly up to 50% and very desirably up to 30%, %s being of the oil phase.

A further class of oils that can be employed herein comprises water-immiscible aliphatic alcohols, and particularly those having a boiling point of higher than 100° C. These include branched chain alcohols of at least 10 carbon atoms and in many instances up to 30 carbon atoms, particularly 15 to 25, such as isostearyl alcohol, hexyl-decanol and octyl-dodecanol. It will be recognised that octyl-dodecanol is an especially favoured water-immiscible aliphatic alcohol in the present compositions, because it not only acts as an emollient oil but additionally moisturises skin by the mechanism of occlusion. Other suitable water-immiscible alcohols include intermediate chain length linear alcohols, commonly containing from 9 to 13 carbon atoms, such as decanol or dodecanol. Such alcohols can often constitute at least 0.1% and particularly at least 0.5% of the oil phase, in many compositions being not more than 5% of the phase.

The present compositions preferably contain a fragrance oil. Such a fragrance oil is normally a complex mixture of chemical classes and accordingly, its proportion is excluded from calculations of the proportion of the other constituents of the liquid oil, including specifically ester, ether and alcohol classes. The total weight proportion of fragrance oils in the emulsion is normally selected in the range of from 0 to 4% of the emulsion, often from 0.3% to 2%.

A solidifying agent is an essential constituent of the oil phase of compositions of the invention. The solidifying agent is preferably a hydrocarbon wax blend, meaning that it consists of various molecules consisting of solely carbon and hydrogen.

Herein, the term “wax blend” or “wax blends” may sometimes be substituted by the term “wax” or “waxes” respectively, without change of meaning.

Preferred hydrocarbon waxes are polyolefin waxes, in particular polyethylene waxes. Such waxes are typically synthetic waxes, i.e. waxes synthesised from olefins in the case of polyolefin waxes and waxes synthesised from ethylene in the case of polyethylene waxes.

Preferred hydrocarbon wax blends that may be used in the present invention have an average molecular weight (M_{w}) of from 450 to 500 and particularly from 460 to 490. Preferred hydrocarbon wax blends may also independently have a polydispersity (M_{w}/Mₙ) of from 1.01 to 1.05, particularly 1.02 to 1.04. A combination of either of the above preferred average molecular weight ranges may be employed with either of the preferred polydispersity ranges to advantageous effect.

The amount of the solidifying agent used in the compositions of the invention is preferably from 5%, more preferably from 7% and most preferably from 8%. The upper amount is preferably up to 25%, more preferably up to 20% and most preferably up to 15%. The preferred ranges of solidifying agent may be selected from any of the lower and upper amounts indicated in this paragraph.

In order to assist the formation and stability of an emulsion, the invention composition comprises at least one emulsifier. In some embodiments, particularly those including a silicone oil, the emulsifier is preferably a dimethicone copolyol having an HLB value of up to 8, particularly from 2 to 7 and especially from 3 to 6. Alkyl dimethicone copolyols are a preferred group of emulsifiers.

In some embodiments, particularly those excluding a silicone oil, the emulsifier preferably comprises polyhydroxylated esters, polyalkylene oxide (including PEG ester, PPG esters and mixed PEG-PPG esters) or polyglyceryl esters. Polyglyceryl esters are particularly preferred, especially in cold processed compositions (vide infra).

Herein, “PEG” refers to polyethylene glycol and “PPG” refers to polypropylene glycol.

The proportion of the emulsifier in the composition is often selected in the range of from 0.125% to 2.0% and in many embodiments from 0.5 to 1.5%.

The compositions herein can incorporate one or more additional components. Such components can include skin feel improvers, such as talc or finely divided high melting point polyethylene (>110° C.), for example in an amount of up to about 6% and often in total in an amount of from 0.5 to 5%; inorganic particulates, preferably finely divided, such as fumed silica, for example in an amount of up to 2%; skin benefit agents, such as allantoin; vitamins or lipids, for example in an amount of up to 5%; colouring agents, such as titanium dioxide; preservatives, such as butylhydroxytoluene, often in an amount of from 0.01 to 0.1%; metal chelates, such as EDTA, for example in an amount of up to 1%; skin cooling agents, such a menthol and menthol derivatives, often in an amount of up to 2%, all of such percentages being of the total composition.

A preferred addition component is a cosmetic skin tone modifier, particularly those selected from niacinamide, 12-hydroxystearic acid, vitamin C, licorice extract and avocado oil. Of these particular materials, niacinamide, vitamin C, licorice extract and avocado oil are temperature sensitive are especially suited to incorporation into the compositions of the invention.

A further optional ingredient can comprise a wash-off agent, which can assist a user of the invention composition to wash it off. Such an agent, when employed, often comprises a nonionic surfactant, such as in an amount of from 0.1 to 2% of the emulsion, and often at least 0.5%. The wash-off agent can be the same as the emulsifier it is preferably a nonionic surfactant such as POE or POP/POE ester or ether (or mixture) having an HLB that is at least 10.

The invention stick compositions can be made in a process comprising: in step 1, making an aqueous phase by blending together the water and water-soluble ingredients, including the ASCH AP active and the polyol, at least a fraction of which may be in the form of a preformed aqueous solution; in step 2, forming an oil phase comprising the solidifying agent and the liquid oil or oil mixture, which normally entails heating the solidifying agent and oils to a temperature at which solidifying agent particles are no longer visible, commonly in the region of the melting point of the solidifying agent; and in a step 3, mixing the aqueous phase and the oil phase together at elevated temperature such that the solidifying agent remains in a liquid state dissolved in the liquid oil, and preferably shear mixing the two phases, in the presence of the emulsifier, and in step 4, filling the resultant mixture into a dispenser whilst it is still mobile and thereafter cooling or allowing the composition to cool, initially until the mixture solidifies and then attains ambient temperature. Herein, this process is known as the “hot process”.

Steps 1 and 2 in the process described above can be carried out simultaneously or sequentially. In step 2, the oil is often heated to a temperature in the region of 70 to 80° C., preferably 70 to 75° C. Before the aqueous phase, prepared in step 1, is mixed with the oil phase in step 3, it preferably heated to temperature in the region of 60 to 70° C., more preferably 65 to 70° C. and most preferably 68 to 69° C. Using these elevated temperatures for the aqueous phase can aid the emulsification process. During step 3, the temperature is maintained at preferably 65 to 80° C., more preferably 65 to 75° C. and most preferably 66 to 74° C.

It is often convenient to incorporate ingredients which are not water-soluble, such as water-insoluble optional ingredients and/or the emulsifier in step 2. If desired, the solidifying agent, or a fraction of them, can be heated externally, for example melted, prior to introduction into the oil phase or emulsion.

If any temperature sensitive ingredients, such as certain fragrance components, are to be added to the compositions, it is often best to add these after step 3 and when the temperature has been reduced, e.g. to less than 70° C., such as from 60 to 68° C.

In a particular embodiment of the process described in the above paragraphs, an antiperspirant composition is manufactured by the steps of (i) making an aqueous phase by blending together water, an ASCH AP active and a polyol as described hereinabove and heating said phase to a temperature of 60° C. or greater; (ii) forming an oil phase comprising the solidifying agent and a liquid oil and heating said phase to a temperature of 70° C. or greater; (iii) with the aqueous phase and oil phase at the temperatures as indicated in steps (i) and (ii) respectively, mixing these phases together in the presence of an emulsifier; (iv) filling the resultant mixture into a dispenser whilst it is still mobile and (v) cooling the composition until the mixture solidifies.

The process described in the paragraph immediately above may be performed by batch or continuous (including semi-continuous) processes.

In a continuous process, the oil and aqueous phases are made separately and typically held with gentle agitation in separate vessels. The oil phase may be held at any suitable temperature, typically within the range 60 to 80° C. The aqueous phase temperature should be adjusted such that when the two phases are combined, the temperature of the mixture is sufficiently high to avoid premature solidification. Typically, this will be in excess of 65° C. The two phases (streams) can then be combined at any point upstream of (or in) a device capable of providing intensive mixing (the primary intensive mixing device) which leads to the formation of the emulsion. Suitable devices include Sonolators and devices where the streams are brought together through impinging jets.

The combined streams can be pumped through the primary intensive mixing device under the required conditions to form the desired emulsion while ensuring that the temperature of the mixture exceeds the solidification temperature. The emulsion formed in this manner may be packed directly into stick packs or sent to a holding vessel where the emulsion droplet size can be maintained or adjusted, by use of a second intensive mixing device (of similar or different design to the primary intensive mixing device) situated either inside the vessel or in a recirculation loop around the vessel, prior to packing the emulsion by filling into stick packs. If desired fragrance may be added to the mixture (emulsion) either as a third stream upstream or downstream of (or in) the primary intensive mixing device or directly into the holding vessel if used.

It is also possible to divide the oil phase into two (or more) streams. This allows the temperatures to which specific formulation components are exposed to be more closely controlled and may be desirable for thermally sensitive components, such as, for example, certain emulsifiers. Several streams may then be combined upstream of or downstream of the primary mixing device, provided that the temperature of the combined system exceeds the solidification temperature.

In an alternative process of manufacture, herein known as the “cold process”, the following steps are followed: (i) making an aqueous phase by blending together water, an ASCH AP active and a polyol as described hereinabove; (ii) forming an oil phase; (iii) mixing the aqueous phase and the oil phase together, preferably at high shear, to form an emulsion at ambient temperature in the presence of an emulsifier; (iv) adding a solidifying agent, in solid form, to the emulsion formed in step (iii) and heating the mixture to a temperature of 70° C. or greater in order to melt/dissolve the solidifying agent; (v) filling the resultant mixture into a dispenser whilst it is still mobile and (vi) cooling the composition until the mixture solidifies.

In a further aspect of the present invention there is provided a stick composition according to the first aspect of the present invention which is contained in solid form in a dispenser. A dispenser for a stick composition often comprises a tubular barrel, having a side wall that surrounds the composition and defines at a first end an opening through which the composition can pass and a second, opposed end within which is fitted a piston, located within the barrel, that is capable of being impelled towards the first end. The means for impelling the piston can comprise a finger or like implement that is pressed by the user against the exposed side of the piston, or a mechanism for imparting axial movement to the piston. Such a mechanism can comprise a threaded spindle that engages with a threaded aperture in the piston, the spindle being mounted on an exposed rotor wheel or fitted with a cog that can be rotated by engagement with a button via a ratchet. The rotor wheel or button is mounted on the barrel adjacent to the second end. Suitable dispensers for firm sticks are described, for example in US 4232977, US4605330, WO09818695, WO09603899, WO09405180, WO09325113, WO09305678, EP1040445, US5997202, US5897263, US5496122, US5275496, US 6598767, US 6299369, or WO 2002/03830.

The present invention includes the non-therapeutic use of the composition according to the invention described herein in order to inhibit or control perspiration, especially in the armpit. In this latter aspect, the composition is wiped across the skin to apply it topically, typically at ambient temperature. The action can be repeated until the user considers that sufficient composition has been deposited, often in the region of 3 to 8 wipes per armpit. The composition is commonly applied shortly after the armpit has been washed or shaved. The composition is thereafter left in place, conventionally, for a period of time commonly between 5 and 24 hours until it is washed off. When seeking to inhibit perspiration, the weight of antiperspirant active applied per armpit is preferably in the range of from 0.15 to 0.5 grams.

Having described the invention and certain preferred embodiments thereof, specific embodiments will now be described more fully by way of example only.

### EXAMPLES

The preparation of Example 1 in Table 1 involved a preliminary step of ‘activating’ the ASCH with calcium chloride and glycine to give “activated ASCH” (AASCH). In this preliminary step, the ASCH, calcium chloride and glycine were heated together with the water for 2 hours at 87° C. The percentage amount for ASCH indicated for Example 1 includes water of hydration. Expressed as anhydrous ASCH, the level used was 12%.

The aqueous solution of AASCH prepared as described above was used to prepare Example 1 by the “hot process” as described hereinabove. Comparative Example A was an emulsion AP stick comprising a typical stick AP active (ZAG) at 20% and this was also prepared by the “hot process”, the aqueous phase used comprising the ZAG, glycerol and water as indicated for this Comparative Example in Table 1.

A Sweat Weight Reduction (SWR) result for Example 1 was obtained in direct comparison with Comparative Example A, using a test panel of 34 female volunteers. Test operators applied ca. 0.3 g of Example 1 to one axilla and Comparative Example A (ca 0.3 g) to the other axilla of each panellist. This was done once each day for three days for each comparison. After the third application, panellists were requested not to wash under their arms for the following 24 hours.

24 hours after the third and final product application, the panellists were induced to sweat in a hot-room at 40° C. (±2° C.) and 40% (±5%) relative humidity, for 40 minutes. After this period, the panellists left the hot-room and their axillae were carefully wiped dry. Preweighed cotton pads were then applied to each axilla of each panellist and the panellists reentered the hot-room for a further 20 minutes. Following this period, the pads were removed and re-weighed, enabling the weight of sweat generated to be calculated.

The SWR for each panellist was calculated as a percentage (% SWR) and the mean % SWR was calculated according to the method described by Murphy and Levine in “Analysis of Antiperspirant Efficacy Results”, J. Soc. Cosmetic Chemists, 1991(May), 42, 167-197.

Example 1 gave a SWR that was 30% greater than Comparative Example A in the head-to-head comparison indicated above. This was a highly significant difference, with a p-value of <0.0001.

TABLE 1

| Component | | Example (followed by percentages by weight) | |
|---|---|---|---|
| Chemical name | Trade name and supplier | 1 | A |
| Aqua | Deionised water | 18.8 | 20 |
| Glycerol | Pricerine 9091 ex Croda | 8 | 8 |
| ASCH | Reach 301, ex Summit | 15 | -- |
| Calcium chloride | Ex Aldrich | 1.5 | -- |
| Glycine | Ex Evonik | 4.7 | -- |
| Zirconium Aluminium Tetrachlorohydrex. GLY | AZG-364, ex Summit | -- | 20 |
| Cetyl PEG/PPG-10/1 Dimethicone | Abil EM90 Ex Evonik | 1.2 | 1.2 |
| Cyclopentasiloxane | DC245 Ex Dow Corning | 19.15 | 19.15 |
| Helianus Anuus Seed Oil | Sunflower seed oil, ex Karlshamns | 3 | 3 |
| C12-15 Alkyl Benzoate | Finsolv TN, ex Evonik | 15.6 | 15.6 |
| Fragrance | Ex Givaudan | 1 | 1 |
| BHT | Ex Eastman | 0.05 | 0.05 |
| Polyethylene wax | Performalene 400, ex New Phase Technologies | 12 | 12 |
| Total: | | 100 | 100 |

Examples 2 and 3 shown in Table 2 were both prepared in analogous manner to Example 1. In these examples, a somewhat lower ratio of glycine to ASCH was used and the amount of

ASCH is expressed as anhydrous ASCH, i.e. excluding water of hydration. The molar ratio of glycine to aluminium in these Examples was 10: 90 and the molar ratio of calcium to aluminium was 9: 91.

SWRs results were obtained for Examples 2 and 3 in comparison with a non-antiperspirant deodorant body spray as a control. The methods used for each test were analogous to that used for the SWR assessment of Example 1. The test performed on Example 2 used a panel of 30 female volunteers and the test performed on Example 3 used a panel of 34 female volunteers. The results from both tests are shown in Table 2 and were highly significant SWR values in favour of the test products.

TABLE 2

| Component | | Example | |
|---|---|---|---|
| Chemical name | Trade name and supplier | 2 | 3 |
| Aqua | Deionised water | 30.146 | 24.116 |
| Glycerol | Pricerine 9091 ex Croda | 12 | 12 |
| ASCH | Reach 301, ex Summit | 12.15 | 9.72 |
| Calcium chloride | Ex Aldrich | 1.516 | 1.213 |
| Glycine | Ex Evonik | 1.188 | 0.95 |
| Cetyl PEG/PPG-10/1 Dimethicone | Abil EM90 Ex Evonik | 1.2 | 1.2 |
| Cyclopentasiloxane | DC245 Ex Dow Corning | 14.8 | 19.2 |
| Helianus Anuus Seed Oil | Sunflower seed oil, ex Karlshamns | 0.1 | 0.1 |
| C12-15 Alkyl Benzoate | Finsolv TN, ex Evonik | 13.8 | 18.4 |
| Fragrance | Ex Givaudan | 1.1 | 1.1 |
| Polyethylene wax | Polarwachs N670, ex Morre-Tec Industries | 12 | 12 |
| SWR (vs. Deo control after 24 hrs.): | | 53% | 47% |

Example 2 as described hereinabove was subjected to large scale consumer test. The test involved 201 female participants, aged 18-55, each of whom had a history of using a stick AP composition at least 5 or 6 times a week and a history of skin sensitivity in the underarm. The participants were each given a stick composition according to Example 2 and were requested to make ‘normal’ in home use of the product for 3 weeks. At the end of the test, the participants were asked to complete a questionnaire.

An analysis of the questionnaires revealed that usage of the sticks during the test was extensive, with 97% of the participants applying the product at least once a day, and of the 201 participants:
153 (76%) responded that the product gave them good, very good or excellent day long protection against wetness;
163 (81%) responded that the product gave them good, very good or excellent day long protection from odour;
168 (84%) responded that the product gave them good, very good or excellent feel of the product on application;
191 (95%) responded that the product gave was good, very good or excellent feel on the criterion of “is kind to skin”;
156 (77%) agreed or strongly agreed with the statement that their skin was smoothed by application of the product.

These results illustrate the ability of compositions of the invention to deliver multiple benefits, including desirable sensory properties and kindness to skin.

In a further consumer test, 226 consumers (including 126 with sensitive skin) were asked whether Example 2 was a better alternative to other deodorants on various criteria. Of the general population (226), 88% judged Example 2 “much more suitable” or “somewhat more suitable” than other deodorants in reducing the symptoms of sensitive skin and 71% judged Example 2 “much better” or “somewhat better” than other deodorants at odour prevention. Of the population with sensitive skin (126), 87% judged Example 2 “much more suitable” or “somewhat more suitable” than other deodorants in reducing the symptoms of sensitive skin and 70% judged Example 2 “much better” or “somewhat better” than other deodorants at odour prevention.

1. A solid antiperspirant stick composition in the form of a water-in-oil emulsion comprising:
(i) an aqueous phase comprising dissolved aluminium sesquichlorohydrate (ASCH) antiperspirant active;
(ii) an oil phase;
(iii) an emulsifier;
(iv) a polyol and
(v) a solidifying agent,
wherein the polyol is glycerol, propylene glycol and/or PEG oligomers having a molecular weight (weight average) of from 180 to 420 and is present at a total level of at least 5% by weight of the total composition and wherein the composition comprises an amino acid and a water-soluble calcium salt.

2. An antiperspirant composition according to claim 1, wherein the polyol is glycerol.

3. An antiperspirant composition according to claim 1_{,} wherein the polyol is present at a total level of at least 8% by weight of the total composition.

4. (canceled)

5. An antiperspirant composition according to claim 1, wherein the amino acid is glycine and the water-soluble calcium salt is calcium chloride.

6. An antiperspirant composition according to claim 1, wherein the ASCH is calcium-activated.

7. An antiperspirant composition according to claim 5, wherein the ASCH AP active is a calcium-activated ASCH glycine complex.

8. An antiperspirant composition according to claim 7, wherein the calcium-activated ASCH glycine complex has a molar ratio of calcium to aluminium that is from 1:15 to 1:5 and a molar ratio of amino acid to aluminium that is also from 1:15 to 1:5.

9. An antiperspirant composition according to claim 1, wherein the ASCH has the formula Al₂OH_{4.7}Cl_{1.3} to Al₂OH_{4.9}Cl_{1.1}.

10. An antiperspirant composition according to claim 1, wherein the aqueous phase constitutes from 50 to 70% by weight of the total composition.

11. An antiperspirant composition according to claim 1, wherein the solidifying agent is a hydrocarbon wax blend.

12. An antiperspirant composition according to claim 1, comprising a cosmetic skin tone modifier.

13. An antiperspirant composition according to claim 1, wherein the oil phase comprises one or more oils selected from volatile silicone oils, ester oils, ether oils or mineral oils.

14. A method of manufacture of an antiperspirant composition comprising the steps of:
(i) making an aqueous phase by blending together water an ASCH antiperspirant active and a polyol and heating said phase to a temperature of 60° C. or greater;
(ii) forming an oil phase comprising a solidifying agent and a liquid oil and heating said phase to a temperature of 70° C. or greater;
(iii) with the aqueous phase and oil phase at the temperatures as indicated in steps (i) and (ii) respectively, mixing these phases together in the presence of an emulsifier;
(iv) filling the resultant mixture into a dispenser whilst it is still mobile and
(v) cooling the composition until the mixture solidifies;
wherein the polyol is glycerol, propylene glycol and/or PEG oligomers having a molecular weight (weight average) of from 180 to 420 and is present at a total level of at least 5% by weight of the total composition and the antiperspirant composition also comprises an amino acid and a water-soluble calcium salt.

15. A non-therapeutic method of reducing perspiration comprising the topical application of a stick composition according to claim 1 .
